(19) United States
(12) Plant Patent Application Publication
(10) Pub. No.: US 2004/0010827 P1
(43) Pub. Date: Jan. 15, 2004

(54) ' HW610' pear

(76) Inventors: David Michael Hunter, St. Catharines (CA); Frank Kappel, Summerland (CA); Harvey Allen Quamme, Penticton (CA); William Gordon Bonn, Windsor (CA)

Correspondence Address: FLYNN, THIEL BOUTELL & TANIS, P.C., 2026 Rambling Road, Kalamazoo, MI, 49008-1699, US

(21) Appl. No.: 10/190,391

(22) Filed: Jul. 3, 2002

(51) Int. Cl. A01H 5/00

(52) U.S. Cl. PLT/176

(57) ABSTRACT

A new and distinct variety of pear tree, which has been given the designation ‘HW610’, which produces a high quality, attractive mid-season fruit and has a good to very good rating for quality of fresh and processed fruit.

### FIELD OF THE INVENTION

The present invention relates to a pear (Pyrus communes L.) variety and more specifically to a mid-season pear variety for the fresh market.

### BACKGROUND OF THE INVENTION

The variety ‘HW610’, developed by Agriculture and Agri-Food Canada Canada at Harrow, Ontario. Plant Breeders Rights was applied for in Canada, application number 00-2184, filed Mar. 28, 2000. This application is still pending. Plant Breeders Rights were applied for the variety in France on Jan. 18, 1995 and were granted as Certificate No. 012246 on Jun. 21, 2001. The variety has also been described by Hunter et al, HortScience, Vol. 37(1):227-229. February 2002. It will be marketed under the brand name AC HARROW CRISP.

### SUMMARY OF THE INVENTION

The new and distinct pear variety, which has been given the designation of ‘HW610’, produces a high quality mid-season pear. The fruits of ‘HW610’ matured at the end of August or early September just after ‘Bartlett’. The fruits are also very attractive with a red blush on a smooth yellow skin. The cream-white flesh is smooth, grit free, and remains firm even when fruits are fully ripe. Fruits of ‘HW610’ have a mild, sweet flavor that is less intense than that of ‘Bartlett’, (patent status unknown). Fruit size on unthinned trees is slightly larger than ‘Bartlett’. It has a good to very good rating for quality of both fresh and processed fruit. The tree of ‘HW610’ is medium in size, conical and upright, annually productive and hardy. The tree has very good fire blight resistance (9.5 rating, similar to ‘Harrow Delight’, patent status unknown) compared to ‘Bartlett’ (4.2 rating). The response to infection follow-na inoculation with the causative organism is more severe in ‘Bartlett’ (mean lesion length 63% of shoot length, with some lesions extending into subtending woody tissues) than in ‘HW610’ (mean lesion 12% of shoot length, maximum lesion length 32% of shoot length).

### BRIEF DESCRIPTION OF THE FIGURE

FIG. 1 is a photographic illustration of the whole fruit of ‘HW610’.

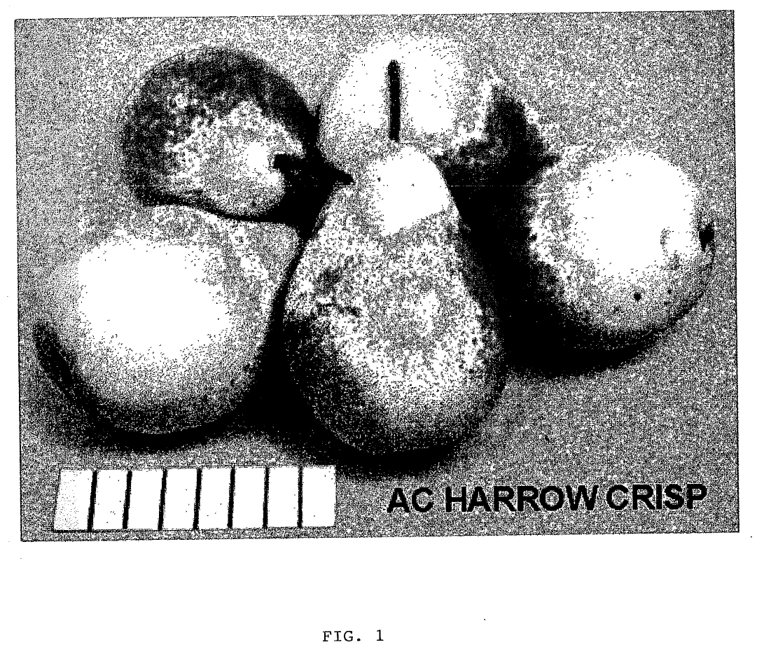

### DETAILED DESCRIPTION OF THE VARIETY

‘HW610’ resulted from a controlled cross between ‘Bartlett’ and US56112-146 made in 1972 by Dr. H. A. Quamme at the Agriculture and Agri-food Canada Research Centre at Harrow, Ontario. It was selected as a hybrid seedling (H7249-A-32) in 1979 by H. A. Quamme, and propagated by budding on pear seedling rootstocks. It was advanced by H. A. Quamme in 1981, and testing began in 1982 at the Harrow Research Centre. Propagation for regional trials in cooperation with the Western Ontario Fruit Testing Association began in 1982, and test trees were placed in regional trials beginning in 1984. Evaluations of second test orchards have been conducted by F. Kappel and D. M. Hunter, and W. G. Bonn has evaluated disease resistance.

Asexual propagation maintains uniformity and stability of ‘HW610’. No variants, off-types or mutants have been observed. The variety will be maintained at the Agriculture and Agri-Food Canada Harrow Research Centre in a virus-tested budwood orchard, and in the holdings of the Canadian Clonal Genebank.

The color terminology is in accordance with The Royal Horticutural Society (RHS) Color Chart.

Tree Habit and Productivity.

The tree of ‘HW610’ is medium in size, conical and upright, annually productive, and winter hardy. In 1994 at Harrow, the original seedling tree and trees grown on ‘Bartlett’ seedling rootstock produced a full crop following winter minimum temperatures as low as −29° C. When propagated on ‘Bartlett’ seedling rootstock, precocity of ‘HW610’ is similar to that of ‘Bartlett’, with bearing initiated within a 4-year period after planting. Annual yields have been greater than those of ‘Bartlett’, especially in areas where fire blight has adversely affected the productivity of ‘Bartlett’. In an evaluation orchard planted in 1982 at Harrow, ‘HW610’ produced commercially acceptable crops until the orchard was removed in Fall 2000 (data not presented), while all ‘Bartlett’ trees had been removed by 1990 because of extensive fire blight damage. ‘HW610’ out-yielded ‘Anjou’ (patent status unknown) and ‘Bosc’, in the first 5 years of an orchard planted at Summerland, B.C. (Table 1).

TABLE 1
Annual fruit yields of ‘HW610’, ‘Anjou’, ‘Bosc’, and
‘Harrow Sweet’ at Summerland, B.C., Canada.^{z}

| | Annual yield (kg/tree) | | | | | Cumulative yield |
|---|---|---|---|---|---|---|
| Cultivar | 1989 | 1990 | 1991 | 1992 | 1993 | (kg/tree) |
| HW610 | 7.6a^{y} | 4.3ab | 18.7a | 12.5a | 21.4ab | 64.5 |
| Anjou | 1.2c | 2.4b | 9.4a | 3.9b | 27.8ab | 44.7 |
| Bosc | 3.2b | 4.5ab | 16.5a | 13.6a | 20.1b | 57.9 |
| Harrow Sweet | 7.7a | 8.1a | 16.4a | 5.4b | 32.7a | 70.3 |

^{Z}Orchard was planted in 1987 with five single tree replicates in a completely randomized design.
^{y}Means separation within columns by Waller-Duncan K ratio t test, P = 0.05. Means within columns followed by the same letter are not significantly different.

Shoot.

The bark on the sun-exposed side of dormant shoots is brown (RHS 165A, 1966). Mean internode length of ‘HW610’ was similar to ‘Bartlett’ and ‘Harrow Sweet’, but shorter than ‘Harvest Queen’ and ‘Bosc’ (Table 2).

TABLE 2
Mean internode length (cm ± SE) as determined
on five successive internodes from the mid-portion of 1-
year-old shoots^{Z}

| | Internode | No. of |
|---|---|---|
| Cultivar | length | Shoots |
| HW610 | 3.1 ± 0.1 | 19 |
| Bartlett | 3.3 ± 0.1 | 13 |
| Harrow Sweet | 3.1 ± 0.1 | 15 |
| Harvest Queen | 3.7 ± 0.2 | 17 |
| Bosc | 4.4 ± 0.1 | 14 |

^{Z}Shoots harvested in February 1996 from mature trees (>10 years old) grown on ‘Bartlett’ seedling rootstock at Harrow, Ont., Canada.

Leaves.

The leaves are elliptic. The shape of the base of the leaf blade is obtuse. The shape of the upper part of the leaf blade is right-angled, with a pointed acuminate tip. There is little curvature of the midrib. Leaf serrations are small and shallow but distinct. The angle between the petiole and the shoot is <30%, the petiole is medium in length, and stipules are absent. The attitude of the leaf in relation to the shoot is upwards. Actively growing shoot tips are reddish-green with light pubescence.

Fire Blight Resistance.

As with other introductions from the Harrow pear breeding program (Hunter et al., 1992, 2002; Quamme and Spearman, 1983], ‘HW610’ (patent status unknown) has excellent resistance to fire blight, similar to or greater than that of ‘Kieffer’ (patent status unknown) which is used as the standard for selection (Hunter, 1993). Using natural fire blight infection scores (van der Zwet et al., 1970), ‘HW610’ had a resistance rating much greater than ‘Bartlett’ (Table 3).

TABLE 3
Ratings of natural and induced fire blight
infections of ‘HW610’ in comparison to ‘Bartlett’,
‘Harrow Sweet’, and ‘Kieffer’.

| | | | |
|---|---|---|---|
| HW610 | Bartlett | Harrow Sweet | Kieffer |

Natural infections^{Z}

| | | | | |
|---|---|---|---|---|
| Rating | 9.5 ± 0.2 | 4.2 ± 0.4 | 9.2 ± 0.2 | 9.3 ± 0.1 |
| Years evaluated | 13 | 18 | 17 | 15 |

Induced infections^{y}

| Lesion (% shoot | 11.8 ± 3.0 | 62.8 ± 4.2 | 14.1 ± 2.5 | 23.1 ± 1.6 |
|---|---|---|---|---|
| length) | | | | |
| Years evaluated | 11 | 22 | 11 | 16 |
| ^{Z}Natural fire blight infections, rated on a scale of 1 (tree dead) to 10 (no blight), are means ± SE of 13 to 18 years at Harrow, Ont., Canada. The rating system was modified from van der Zwet et al. (1970) by assigning values of 10 = no visible blight and 9 =< 3% infection. For ‘HW610’ and ‘Harrow Sweet’, ratings were made on the own-rooted seedling tree. For ‘Bartlett’ and ‘Kieffer’, ratings were made on trees grown on | | | | |
| # ‘Bartlett’ seedling rootstock in a nearby (within 200 m) cultivar evaluation orchard. In both the seedling orchard and the cultivar evaluation orchard, susceptible trees had severe fire blight infections each year. | | | | |

^{y}Induced infections were rated in late July, ≈5 weeks after inoculating 10-20 actively growing shoots with 20 μL of a cocktail of six virulent strains of Erwinia amylovora (10⁸ cfu/mL). Values indicate lesion length expressed as a percentage of total shoot length. Data are means ± SE of 11 to 22 years.

When actively growing shoot tips were inoculated with a mixture of six virulent strains of E. amylovora, the length of the lesion that developed extended to ≈12% of current season's growth, similar to ‘Harrow Sweet’, patented, and ‘Kieffer’, but much less than lesion development in ‘Bartlett’ (Table 3).

Bloom and Pollination.

At Harrow, the time of full bloom of ‘HW610’ is similar to that of ‘Bartlett’. First bloom, however, is 2 d later than ‘Bartlett’. Flower clusters typically contain seven flowers, occasionally six or eight, rarely five or nine. Petals are white, almost as long as broad, and just touch with no overlap. The pink to red anthers are large in size, and are level with or slightly above the stigma. ‘HW610’ tends to be a poor pollinizer.

In controlled pollination tests, fruit set was used to determine pollen compatibility when pollen from a known source was applied to stigmatic surfaces immediately after emasculation of the flower. Because emasculated pear flowers are even less attractive to bees and other pollinating insects than non-emasculated flowers, bagging was not considered necessary. ‘HW610’ will not consistently pollinate ‘Bartlett’, ‘Bosc’, or ‘Anjou’, especially when spring weather is warm and humid; however, under the cooler conditions experienced in Spring 2000, ‘HW610’ pollinated ‘Bartlett’, ‘Bosc’, ‘Anjou’, “Flemish Beauty’, and ‘HW616’, but these cultivars did not adequately pollinate ‘HW610’. Also in 2000, pollination of emasculated flowers of ‘HW610’ with pollen of ‘HW610’ resulted in adequate fruit set for commercial production. Seed set in ‘HW610’ tends to be low, and large-sized fruits can develop with few or no viable seeds.

### Fruit Characteristics

Size, Shape, and Color.

Fruits are slightly larger than ‘Bartlett’ (Table 4).

TABLE 4
Harvest dates at Harrow, Ont., Canada, and
fresh fruit evaluations for ‘Bartlett’, ‘HW610’, and
‘Harrow Sweet’

| | Bartlett | HW610 | Harrow Sweet |
|---|---|---|---|
| Years evaluated | 20 | 17 | 18 |
| Harvest dates | | | |
| Average | 28 Aug. | 3 Sept. | 18 Sept. |
| Earliest | 18 Aug. | 22 Aug. | 29 Aug. |
| Latest | 9 Sept. | 27 Sept. | 8 Oct. |
| Size (mm) | | | |
| Length | 81.4a^{Z} | 84.4a | 84.6a |
| Diameter | 63.4a | 65.1a | 63.5a |
| Ratings^{y} | | | |
| Appearance^{X} | 7.7b | 8.6a | 7.4b |
| Flavor^{X} | 8.0a | 7.4b | 7.8ab |
| Texture^{X} | 7.9a | 7.4ab | 7.1b |
| Weighted score^{W} | 81.3a | 79.4ab | 77.1b |
| Grit^{V} | 3.9a | 4.2a | 3.1b |
| Juice^{U} | 3.9a | 3.1b | 4.2a |
| Core^{t} | 3.2b | 3.0b | 3.8a |

^{Z}Means separation within rows by Duncan's new multiple range test, P = 0.05. Means within rows followed by the same letter are not significantly different.
^{y}Ratings reported are based on evaluations of fruits ripened immediately after harvest.
^{X}Appearance, flavor and texture ratings [on a scale of 1 (least desirable) to 9 (most desirable)] were determined each year by two to four trained panelists.
^{W}Weighted score = (3 × appearance) + (5 × flavor) + (2 × texture).
^{V}Grit rating is on a scale of 1 (undesirable, i.e. large and/or many grit cells) to 5 (desirable, i.e., very small and/or few or no grit cells).
^{U}Juiciness rating is on a scale of 1 (dry) to 5 (very juicy).
^{t}Core size rating is on a scale of 1 (small) to 5 (large).

Fruit shape is symmetrical, pyriform, and concave to almost straight in profile. Using International Board for Plant Genetic Resources descriptors (Thibault et al., 1983), the predominant fruit shape has been described as 5.2 (≈50% of individual fruits), while other individual fruits have been described as 5.4 (≈25%) or 7.2 (≈20%). The calyx is persistent at harvest, with short to medium length sepals that are convergent to upright. The calyx basin is medium depth and narrow to medium in width, and the margin is even to slightly ribbed. Following ripening at ≈20° C., the skin has a very attractive golden yellow ground color (RHS 11A or 11B) with a red blush on the sun-exposed fruit surface. The skin is very smooth and there is little or no russeting of the fruit. The flesh is white to cream-white in color, very fine in texture, grit-free, and remains firm even when fully ripe. Fruits have a mild sweet flavor and relatively little juice.

Maturity.

At Harrow, Ont., Canada, the fruits of ‘HW610’ mature at the end of August or early September, just after ‘Bartlett’ (Table 4), and can be harvested over a 2-week period Early picked fruits can be stored in common cold storage (1 to 2° C.) for ≈2 months, but storage life is reduced with later picking. Core breakdown can be a problem with overripe fruits.

Quality.

At Harrow, fruits were harvested each year at the normal fresh market maturity for commercial harvest (5-7 kg pressure). Following ripening at ≈20° C. until ‘eating ripe’, a sample of 5-10 fruits, selected at random, was evaluated for appearance, flavor, texture, number and size of grit (stone cells) in the flesh, juiciness, and core size relative to fruit size. Evaluations were made on fruits ripened immediately after harvest. At Harrow, trained panelists rated the appearance of ripened fruits of ‘HW610’ as excellent, and significantly better than ‘Bartlett’ and ‘Harrow Sweet’. Fresh fruit quality of ‘HW610’, as indicated by the weighted score, was intermediate between, but not statistically different from, ‘Bartlett’ and ‘Harrow Sweet’ (Table 4). There are no significant differences between fruits ripened immediately after harvest and fruits ripened after 4 weeks in a common cold storage at ≈2° C. (data not presented).

Processing Evaluations.

When ripened fruits are processed as pear halves, ‘HW610’ remains intact, with virtually no breakdown, and there is no discoloration of the fruit or syrup. ‘HW610’ rates almost as highly as ‘Bartlett’, and significantly better than ‘Harrow Sweet’ (Table 5).

TABLE 5
Ratings of pear halves^{Z} and pear purée^{y}
processed from ripened fruits of ‘HW610’ in comparison
with ‘Bartlett’ and ‘Harrow Sweet’.

| | HW610 | Bartlett | Harrow Sweet |
|---|---|---|---|
| Halves | 3.8a^{X} | 3.9a | 3.4b |
| Puree | 3.6b | 4.0a | 3.3b |
| ^{Z}Determined with masked identity taste panels involving four trained panelists. Fruits were processed in syrup containing 15% (w/v) sugar. Processing rating for pear halves is the average of ratings for flavor, texture and appearance on a scale of 1 (least desirable) to 5 (most desirable). Samples, including a masked identify ‘Bartlett’ sample, were compared to a known ‘Bartlett’ sample. | | | |
| # Data presented are means of 5 years. | | | |

^{y}Determined with masked identify taste panels involving four trained panelists. Fruits were processed with no additional sugar. Processing rating for purée is the average of ratings for viscosity, color, and flavor on a scale of 1 (least desirable) to 5 (most desirable). Samples, including a masked identify ‘Bartlett’ sample, were compared to a known ‘Bartlett’ sample. Data presented are means of 5 years.
^{X}Means separation within rows by Duncan's new multiple range test, P = 0.05. Means within rows followed by the same letter are not significantly different.

When processed as pear purée, ‘HW610’ is rated better than ‘Harrow Sweet’ but not as high as ‘Bartlett’. While the processed product from small scale trials has been rated good, the quality may not be sufficiently high for ‘HW610’ to have commercial acceptability for processing as halves or purée in the current market.

‘HW610’ was tested at the Canadian Centre for Plant Health, Saanichton, B.C., using woody-host and herbaceous-host biological indicators, and by serological and molecular methods, and found to be free of all known viruses, virus-like agents, viroids, and phytoplasmas. Virus-tested trees have been planted in the Canadian Clonal Gene Bank at Harrow.

The claim:

1. A new and distinct pear variety substantially as shown and described herein.
